SEQUENCE LISTING (16 sequences)

SEQ ID NO: 1 - DNA, 18 bp, Artificial Sequence
  synthetic primer
tggaatgcgagtgcctag

SEQ ID NO: 2 - DNA, 20 bp, Artificial Sequence
  synthetic primer
accgtcctgctgtctatatc

SEQ ID NO: 3 - DNA, 19 bp, Artificial Sequence
  synthetic primer
cgtggaactggcagaagag

SEQ ID NO: 4 - DNA, 20 bp, Artificial Sequence
  synthetic primer
gtagacagaagagcgtggtg

SEQ ID NO: 5 - DNA, 19 bp, Artificial Sequence
  synthetic primer
cttggtagaggtgactgag

SEQ ID NO: 6 - DNA, 21 bp, Artificial Sequence
  synthetic primer
aagttgtagactgttaaggtc

SEQ ID NO: 7 - DNA, 18 bp, Artificial Sequence
  synthetic primer
cagcatcacctacgacac

SEQ ID NO: 8 - DNA, 19 bp, Artificial Sequence
  synthetic primer
ggctctgtaacttccttgg

SEQ ID NO: 9 - DNA, 18 bp, Artificial Sequence
  synthetic primer
gccctttcggagctgaag

SEQ ID NO: 10 - DNA, 20 bp, Artificial Sequence
  synthetic primer
ttaactgacagccagccaag

SEQ ID NO: 11 - DNA, 22 bp, Artificial Sequence
  synthetic primer
ccgtaaagacctctatgccaac

SEQ ID NO: 12 - DNA, 20 bp, Artificial Sequence
  synthetic primer
acatctgctggaaggtggac

SEQ ID NO: 13 - DNA, 20 bp, Artificial Sequence
  synthetic primer
tctctgggaaatcgtggaaa

SEQ ID NO: 14 - DNA, 23 bp, Artificial Sequence
  synthetic primer
ccaggtagctatggtactccaga

SEQ ID NO: 15 - DNA, 22 bp, Artificial Sequence
  synthetic primer
agagggattgtgtcacttcgtt

SEQ ID NO: 16 - DNA, 19 bp, Artificial Sequence
  synthetic primer
gcatatgaagagggcagca

(19) United States
(12) Patent Application Publication
(10) Pub. No.: US 2010/0267619 A1
(43) Pub. Date: Oct. 21, 2010

(54) Methods and Compositions for Treatment of Atherosclerosis

(76) Inventor: PATRICE DELAFONTAINE, NEW ORLEANS, LA (US)

Correspondence Address: Tulane University;Mr. Jacob Maczuga, 6823 St. Charles Avenue, New Orleans, LA, 70118, US

(21) Appl. No.: 12/697,014

(22) Filed: Jan. 29, 2010

Related U.S. Application Data
(63) Continuation of application No. 11/880,401, filed on Jul. 19, 2007, now abandoned.
(60) Provisional application No. 60/832,330, filed on Jul. 21, 2006.

(51) Int. Cl. A61K 38/30; A61P 9/10

(52) U.S. Cl. 514/1.9

(57) ABSTRACT

The present invention provides methods and compositions for treating subject having atherosclerosis. The invention provides a method comprising administering to a subject having atherosclerosis an effective amount of insulin like growth factor-1 (IGF-1), wherein the administering is effective to treat atherosclerosis in the subject. The invention also provides kits for practicing the methods of the invention.

### CROSS-REFERENCE

This application claims the benefit of U.S. Provisional Patent Application No. 60/832,330, filed Jul. 21, 2006, which application is incorporated herein by reference in its entirety.

### GOVERNMENT RIGHTS

This invention was made with government support under U.S. federal grant nos. R01 HL070241 and R01 HL080682 awarded by the National Institutes of Health. The United States Government may have certain rights in this invention.

### BACKGROUND OF THE INVENTION

Atherosclerosis, the most common form of vascular disease, is the disorder of large arteries that underlies most coronary artery disease, aortic aneurysm, and arterial disease of lower extremities, and is believed to play a major role in cerebrovascular disease (Libby, in “The Principles of Internal Medicine”, 15th ed., Braunward et al. (editors), Saunders, Philadelphia, Pa., 2001, pp. 1377-1382.). One theory for pathogenesis of atherosclerosis that is consistent with a variety of experimental evidence is the “reaction to injury” hypothesis (Libby, in “The Principles of Internal Medicine”, 15th ed., Braunward et al. (editors), Saunders, Philadelphia, Pa., 2001, pp. 1377-1382). The injury to the endothelium may be subtle, resulting in a loss of the ability of the cells to function normally. Examples of types of injury to the endothelium include hypercholesterolemia and mechanical stress (Ross, 1999, N. Engl. J. Med., 340: 115).

In general, atherosclerosis can affect the medium-sized and large arteries of the brain, heart, kidneys, other vital organs, and legs and results in the wall of an artery becoming thicker and less elastic. There are two main theories about why atherosclerosis develops. The first theory is that high levels of cholesterol in the blood injure the artery's lining, causing an inflammatory reaction and enabling cholesterol and other fatty materials to accumulate there. The second theory is that repeated injury to the artery's wall may occur through various mechanisms involving the immune system or through direct toxicity. In both cases, there are changes that can lead to the formation of atheromas (plaques deposits).

Atherosclerosis is thought to also involve inflammation, because certain white blood cells—lymphocytes, monocytes, and macrophages—are present throughout the development of atherosclerosis. These cells usually gather only when inflammation develops. Atherosclerosis begins when monocytes are activated and move out of the bloodstream into the wall of an artery. There, they are transformed into foam cells, which collect cholesterol and other fatty materials. In time, these fat-laden foam cells accumulate and form atheromas in the lining of the artery's wall, causing a thickening and hardening of the wall. Atheromas may be scattered throughout medium-sized and large arteries, but usually form where the arteries branch—presumably because the constant turbulent blood flow at these areas injures the artery's wall, making these areas more susceptible to atheroma formation.

While the administration of anti-inflammatory agents and lipid altering compounds has been suggested in therapy for atherosclerosis, little success has been reported.

### LITERATURE

U.S. Pat. Nos. 5,843,899, 6,485,480; U.S. Patent Application Published No. 2003/0060415; Ross et al., N Engl J Med. 1999; 340:115-26; Libby et al., Nature. 2002; 420:868-74; Juul et al., Circulation. 2002; 106:939-44; Vasan et al., Ann Intern Med. 2003; 139:642-8; Scheidegger et al., J Biol Chem. 2000; 275:26864-9; Delafontaine et al., Arterioscler Thromb VascBiol. 2004; 24:435-44; Li et al., Arterioscler Thromb Vase Biol. 2003; 23:2178-84; Okura et al., Circulation. 2000; 102:2680-6; Nakashima et al., Arterioscler Thromb. 1994; 14:133-40; Barath et al., Am J Cardiol. 1990; 65:297-302; Rus et al., Atherosclerosis. 1996; 127:263-71; Tzoulaki et al., Circulation. 2005; 112:976-83; Hwang et al., Circulation. 1997; 96:4219-25; Rauscher et al., Circulation. 2003; 108:457-63; Asahara et al., Science. 1997; 275:964-7; Goldschmidt-Clermont et al., Circulation. 2005; 112:3348-53; Conti et al., Circulation. 2004; 110:2260-5; Kiely et al., J Biol Chem. 2002; 277:22581-9; Isenovic et al., Metabolism. 2003; 52:482-7; Aicher et al., Nat Med. 2003; 9:1370-6; Xu et al., Circ Res. 2003; 93:e76-86; Kalka et al., Proc Natl Acad Sci USA. 2000; 97:3422-7; Urbich et al., J Mol Cell Cardiol. 2005; 39:733-42; Shi et al., Blood. 1998; 92:362-7; Huber et al., Arterioscler Thromb Vase Biol. 1999; 19:2364-7; Spies et al., Gene Ther. 2001; 8:1409-15; Lovett-Racke et al., J Clin Invest. 1998; 101:1797-804; Kaminski et al., Am J Pathol. 2004; 164:2241-9; Li et al., Arterioscler Thromb Vasc Biol. 2002; 22:249-55; Kunjathoor et al., J Clin Invest. 1996; 97:1767-73; Li et al. (Nov. 15, 2005) American Heart Assn.

### SUMMARY OF THE INVENTION

The present invention provides methods and compositions for treating subject having atherosclerosis. The invention provides a method comprising administering to a subject having atherosclerosis an effective amount of insulin like growth factor-1 (IGF-1), wherein the administering is effective to treat atherosclerosis in the subject. The invention also provides kits for practicing the methods of the invention.

The present invention features a method for treating a subject having atherosclerosis by administering to a subject having atherosclerosis an effective amount of insulin like growth factor-1 (IGF-1), wherein the administering is effective to treat atherosclerosis in the subject. In some embodiments, the IGF-1 is administered in a dose of about 10 to 400 μg/kg/day. In further embodiments, the IGF-1 is administered in a dose of about 20 to 240 μg/kg/day.

In some embodiments, the IGF-1 is provided in a sustained release formulation. In some embodiments, the IGF-1 is administered systemically to the subject. In some embodiments, the IGF-1 is administered subcutaneously. In certain embodiments, the IGF-1 is administered to the subject by bolus injection. In some embodiments, the IGF-1 is administered by a mini-pump. In certain embodiments, the IGF-1 is administered in a rate of about 10 μg/kg/day to 400 μg/kg/day. In further embodiments, the IGF-1 is administered systemically to the subject in a sustained release formulation.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 is a graph showing the measurement of lesion areas in IGF-1 infusion and sham infusion ApoE^{-/-} mice as a percentage of total aortic area (P<0.01 compared IGF-1 infusion (n=27) and sham infusion (n=29)).

FIG. 2 is a series of graphs showing effect of IGF-1 on levels of circulating IL-6 (Panel A) and TNF-α (Panel B) as determined by ELISA (P<0.001 (n=32)).

FIGS. 3A and 3B depict the effect of IGF-1 on atherosclerotic plaque progression and macrophage accumulation in aortic valves of ApoE-deficient mice.

FIGS. 4A-C depict the effect of IGF-1 on oxidative stress in vivo and in cultured endothelial cells.

### DEFINITIONS

Before describing the invention in greater detail, the following definitions are set forth to illustrate and define the meaning and scope of the terms used to describe the invention herein.

As used herein, “atherosclerosis” refers to a disease of the arterial blood vessels that results in hardening or furring of the arteries caused by the formation of multiple atheromatous plaques within the arteries. Pathologically, the atheromatous plaque includes nodular accumulation of a soft, flaky, yellowish material at the center of large plaques, composed of macrophages nearest the lumen of the artery, sometimes with underlying areas of cholesterol crystals, and possibly also calcification at the outer base of older/more advanced lesions. The atheromatous plaques, though compensated for by artery enlargement, eventually lead to plaque ruptures and stenosis (i.e., narrowing) of the artery and, therefore, an insufficient blood supply to the organ it feeds. Alternatively, if the compensating artery enlargement process is excessive, then a net aneurysm results. The complications associated with atherosclerosis are chronic, slowly progressing and cumulative. Most commonly, the rupture of a soft plaque causes the formation of a blood clot (e.g., thrombus) that will rapidly slow or stop blood flow, e.g. 5 minutes, leading to death of the tissues fed by the artery. A common recognized scenario is coronary thrombosis of a coronary artery causing myocardial infarction (i.e., a heart attack). Another common scenario in advanced disease is claudication from insufficient blood supply to the legs, typically due to a combination of both stenosis and aneurysmal segments narrowed with clots. Kidney, intestinal and other arteries are also typically involved.

As used herein, “subject,” “individual,” or “patient” refers to any mammal, including humans, bovines, ovines, porcines, canines and felines, in need of treatment. In certain embodiments, the patient is a human. In general, the methods of the invention are applicable to pediatric and adult patients.

The term “concentration in blood,” such as in the phrases “IGF-1 concentration in blood”, refers to a concentration of an agent (e.g., IGF-1) obtained in whole blood or in a fluid obtained from blood, such as plasma or serum.

As used herein, “IGF-1” refers to insulin-like growth factor-1 from any species, including bovine, ovine, porcine, equine, avian, and human, in native-sequence or in variant form, and from any source, whether natural, synthetic, or recombinant.

Suitable for use in the subject methods is human native-sequence, mature IGF-1, for example, without an N-terminal methionine, prepared, e.g., by the process described in EP 230,869 published Aug. 5, 1987; EP 128,733 published Dec. 19, 1984; or EP 288,451 published Oct. 26, 1988. In some embodiments, this native-sequence IGF-1 is recombinantly produced as described in EP 123,228 and 128,733, and U.S. Pat. No. 6,331,414 issued Dec. 18, 2001. In other embodiments, this native-sequence IGF-1 is the active pharmaceutical ingredient in the drug product commercially marketed as INCRELEX™ (mecasermin [rDNA origin] for injection). The term “rhIGF-1” refers to recombinant human IGF-1.

As used herein, reference to “variants” or “analogs, homologs and mimics” of IGF-1 embraces compounds which differ from the structure of native IGF-1 by as little as the replacement and/or deletion of one or more residues thereof, to compounds which have no apparent structural similarity. Such compounds in all instances, however, have substantially the same activity as native IGF-1. Thus, “analogs” refers to compounds having the same basic structure as IGF-1, but differing in several residues; and “homologs” refers to compounds which differ from native IGF-1 by the deletion and/or replacement of a limited number of residues.

Suitable for use in the present invention are IGF-1 variants described in U.S. Pat. Nos. 5,077,276 issued Dec. 31, 1991; 5,164,370; 5,470,828; in PCT WO 87/01038 published Feb. 26, 1987 and in PCT WO 89/05822 published Jun. 29, 1989, i.e., those wherein at least the glutamic acid residue is absent at position 3 from the N-terminus of the mature molecule or those having a deletion of up to five amino acids at the N-terminus. In some embodiments, the variant has the first three amino acids from the N-terminus deleted (variously designated as brain IGF, tIGF-1, des(1-3)-IGF-1, or des-IGF-1). Other compounds are the IGF-1 displacers compounds as described below, and in U.S. Pat. Nos. 6,121,416, 6,251,865, and 6,420,518.

As used herein, an “IGF binding protein” or “IGFBP” refers to a protein or polypeptide normally associated with or bound or complexed to IGF-1 or IGF-2, whether or not it is circulatory (i.e., in blood (e.g., serum) or tissue). Such binding proteins do not include receptors. This definition includes IGFBP-1, IGFBP-2, IGFBP-3, IGFBP-4, IGFBP-5, IGFBP-6, Mac 25 (IGFBP-7), and prostacyclin-stimulating factor (PSF) or endothelial cell-specific molecule (ESM-1), as well as other proteins with high homology to IGFBPs. Mac 25 is described, for example, in Swisshelm et al., 1995, Proc Natl Acad Sci USA, 92: 4472-4476 and Oh et al., J Biol Chem, 271: 30322-30325 (1996). PSF is described in Yamauchi et al., 1994, Biochem J, 303:591-598. ESM-1 is described in Lassalle et al., 1996, J Biol Chem, 271: 20458-20464. For other identified IGFBPs, see, e.g., EP 375,438 published Jun. 27, 1990; EP 369,943 published May 23, 1990; WO 89/09268 published Oct. 5, 1989; Wood et al., 1988, Mol Endocrinol, 2: 1176-1185; Brinkman et al., 1988, EMBO J, 7: 2417-2423; Lee et al., 1988, Mol Endocrinol, 2:404-411; Brewer et al., 1988, Biochem Biophys Res Comm, 152: 1289-1297; EP 294,021 published Dec. 7, 1988; Baxter et al., 1987, Biochem Biophys Res Comm, 147: 408-415; Leung et al., 1987, Nature, 330: 537-543; Martin et al., 1986, J Biol Chem, 261:8754-8760; Baxter et al., 1988, Comp Biochem Physiol, 91B: 229-235; WO 89/08667 published Sep. 21, 1989; WO 89/09792 published Oct. 19, 1989; and Binkert et al., 1989, EMBO J, 8: 2497-2502.

As used herein, “active”, “bioactive”, “biologically active” or “free” IGF-1 in the context of changing blood and tissue levels of endogenous IGF-1 refers to IGF-1 that binds to an IGF receptor or an insulin receptor, or a hybrid IGF/insulin receptor, or to an IGF binding protein, or otherwise causes a biological activity of endogenous or exogenous IGF-1 to occur.

As used herein, “treatment” or “treating” refers to inhibiting the progression of a disease or disorder, e.g., atherosclerosis, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect may be prophylactic in terms of completely or partially preventing a disease or condition, or a symptom thereof and/or may be therapeutic in terms of a partial or complete cure for a disease or disorder and/or adverse affect attributable to the disease or disorder. “Treatment,” as used herein, covers any treatment of a disease or disorder in a mammal, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of disease or conditions associated with atherosclerosis.

As used herein, a “therapeutically effective amount” refers to that amount of the compound sufficient to treat or manage a disease or disorder, e.g., short stature or other endocrine disorder characterized by partial endogenous growth hormone activity or signaling. A therapeutically effective amount may refer to the amount of a compound that provides a therapeutic benefit in the treatment or management of a disease or disorder. Further, a therapeutically effective amount with respect to a compound of the invention means that amount of compound alone, or in combination with other therapies, that provides a therapeutic benefit in the treatment or management of a disease or disorder. The term can encompass an amount that improves overall therapy, reduces or avoids unwanted effects, or enhances the therapeutic efficacy of or synergies with another therapeutic agent.

As used herein, a “pharmaceutical composition” is meant to encompass a composition suitable for administration to a subject, such as a mammal, especially a human. In general a “pharmaceutical composition” is sterile, and free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, subcutaneous, intradermal, intratracheal and the like. In some embodiments the composition is suitable for administration by a transdermal route, using a penetration enhancer other than dimethyl sulfoxide (DMSO). In other embodiments, the pharmaceutical compositions are suitable for administration by a route other than transdermal administration.

As used herein, the phrase “pharmaceutically acceptable carrier” refers to a carrier medium that does not interfere with the effectiveness of the biological activity of the active ingredient. Said carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is an example of a suitable carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour; chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes are suitable carriers for gene therapy uses of the invention. Such pharmaceutical compositions should contain a therapeutically effective amount of the compound, together with a suitable amount of carrier so as to provide the form for proper administration to the subject. The formulation should suit the mode of administration.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

As used herein, the phrase “pharmaceutically acceptable salts” refers to salts prepared from pharmaceutically acceptable, essentially nontoxic, acids and bases, including inorganic and organic acids and bases. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

As used herein, an “immediate release” formulation of IGF-1 refers to a drug composition or mixture of drug compositions in which there is no carrier that regulates the bioavailability of the drug's active ingredient(s) to tissues at the site of drug administration in the patient's body. It will be understood that any component of the formulation that limits or impairs access of the drug's active ingredient(s) to tissues at the site of drug administration in the patient's body is a carrier that regulates the bioavailability of the active ingredient(s) so affected for purposes of the foregoing definition. By this definition, a formulation comprising an IGF-1/IGFBP-3 non-covalently associated complex fails to qualify as an “immediate release” formulation of IGF-1. Since it prevents the IGF-1 in the complex from binding to IGF-1 receptors in tissues and/or competes with IGF-1 receptors in tissues for binding to such IGF-1, the IGFBP-3 component of the IGF-1/IGFBP-3 non-covalently associated complex limits or impairs the access of such IGF-1 to tissues, and so qualifies as a carrier that regulates the bioavailability of such IGF-1 to tissues at the site of drug administration in the patient's body. In addition, this definition precludes any formulation that provides sustained-release or time-release of the drug's active ingredient(s) from a pool or reservoir in a macromolecular matrix carrier. For example, a formulation comprising IGF-1 encapsulated in a bioerodible microsphere carrier fails to qualify as an “immediate release” formulation of IGF-1.

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “an individual” includes one or more individuals, and reference to “the method” includes reference to equivalent steps and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citation or discussion of a reference herein shall not be construed as an admission that such is prior art to the present invention.

The invention will now be described in more detail.

### DETAILED DESCRIPTION OF HE INVENTION

The present invention provides methods and compositions for treating subject having atherosclerosis. The invention provides a method comprising administering to a subject having atherosclerosis an effective amount of insulin like growth factor-1 (IGF-1), wherein the administering is effective to treat atherosclerosis in the subject.

Subjects suitable for treatment with the methods disclosed herein include subjects that suffer from atherosclerosis, particularly those having or at risk of an atherosclerotic disease event, such as stroke, acute coronary syndrome including heart attack, heart disease including congestive heart failure, peripheral artery occlusive disease, and the like.

Atherosclerosis is characterized by the deposition of atheromatous plaques containing cholesterol and lipids on the innermost layer of the walls of large and medium-sized arteries. Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

The term “atherosclerotic disease event” as used herein is intended to encompass disease events arising from complications associated with atherosclerosis, including but not limited to, coronary heart disease events, cerebrovascular events, an acute coronary syndrome, and intermittent claudication. For example, atherosclerosis of the coronary arteries commonly causes coronary artery disease, myocardial infarction, coronary thrombosis, and angina pectoris. Atherosclerosis of the arteries supplying the central nervous system frequently provokes strokes and transient cerebral ischemia. In the peripheral circulation, atherosclerosis causes intermittent claudication and gangrene and can jeopardize limb viability. Atherosclerosis of an artery of the splanchnic circulation can cause mesenteric ischemia. Atherosclerosis can also affect the kidneys directly (e.g., renal artery stenosis). It is intended that persons who have previously experienced one or more non-fatal atherosclerotic disease events are those for whom the potential for recurrence of such an event exists.

Acute coronary syndrome represents a form of acute destabilization of atherosclerotic plaques often caused by plaque rupture that results in acute myocardial ischemia. Acute myocardial ischemia is chest pain due to insufficient blood supply to the heart muscle that results from coronary artery disease (also called coronary heart disease). Patients who have symptoms of acute coronary syndrome may or may not exhibit an ST elevation (also referred to as an ST displacement) by electrocardiogram (ECG or EKG), which is diagnostic of damage to the cardiac muscle or strain on the ventricles. Most patients who exhibit ST-segment elevation in an ECG ultimately develop a Q-wave acute myocardial infarction (i.e., heart attack). Patients who have ischemic discomfort without ST-segment elevation are generally diagnosed as having unstable angina or a non-ST-segment elevation myocardial infarction (the latter of which can lead to a non-Q-wave myocardial infarction). Acute coronary syndrome thus encompasses the spectrum of clinical conditions ranging from unstable angina to non-Q-wave myocardial infarction and Q-wave myocardial infarction.

The method of this invention particularly serves to slow new atherosclerotic lesion or plaque formation, and to slow progression, including stopping progression, of existing lesions or plaques, as well as to cause regression of existing lesions or plaques. In addition, this intervention may accelerate healing of unstable or ruptured plaque.

The methods of the invention contemplate methods for slowing the progression, including stopping progression, of atherosclerosis, including slowing atherosclerotic plaque progression, comprising administering a therapeutically effective amount of IGF-1 to a patient in need of such treatment. This method also includes slowing progression, including stopping progression, of atherosclerotic plaques existing at the time the instant treatment is begun (i.e., “existing atherosclerotic plaques”), as well as halting or stopping formation of new atherosclerotic plaques in patients with atherosclerosis.

The methods disclosed herein also encompass methods for regression of atherosclerosis, including regression of atherosclerotic plaques existing at the time the instant treatment is begun, comprising administering a therapeutically effective amount of IGF-1 to a patient in need of such treatment.

In addition, the methods disclosed herein also encompass methods for slowing atherosclerotic plaque progression and/or accelerating plaque healing so as to reduce the risk of atherosclerotic plaque rupture comprising administering a prophylactically effective amount of IGF-1 to a patient in need of such treatment, e.g. acute syndrome associated with impending plaque rupture. Rupture as used herein refers to rupture of an atherosclerotic plaque often at the site of a thin fibrous cap, which potentially leads to thrombus formation and an acute event. A further aspect of this invention involves a method for preventing or reducing the risk of developing atherosclerosis, comprising administering a prophylactically effective amount of the compounds described herein to a patient in need of such treatment.

Subject suitable for treatment according to the methods of the invention include those who a medical practitioner has diagnosed as having one or more symptoms of atherosclerosis, and particularly those patients who have had or are at risk of an atherosclerotic disease event. Diagnosis may be done by any suitable means. Methods for diagnosing atherosclerosis by measuring systemic inflammatory markers are described, for example, in U.S. Pat. No. 6,040,147, hereby incorporated by reference. Diagnosis and monitoring may employ an electrocardiogram, chest X-ray, cardiac catheterization, ultrasound (for the measurement of vessel wall thickness), or measurement of blood levels of CPK, CPK-MB, myoglobin, troponin, homocysteine, or C-reactive protein.

One in the art will understand that a patient at risk of development of an atherosclerotic disease event may have been subjected to the same tests (electrocardiogram, chest X-ray, etc.) or may have been identified, without examination, as one at high risk due to the presence of one or more risk factors (e.g., family history, hypertension, diabetes mellitus, high cholesterol levels, smoking, obesity, etc.).

Usually, atherosclerosis does not produce symptoms until it narrows the interior of an artery by more than 70%. Symptoms depend on location of the narrowing or blockage, which can occur almost anywhere in the body. Symptoms occur because as atherosclerosis narrows an artery more and more, tissues supplied by the artery may not receive enough blood and oxygen. The first symptom of a narrowing artery may be pain or cramps at times when blood flow cannot keep up with the tissues' need for oxygen. Typically, symptoms develop gradually as the atheroma slowly narrows an artery. However, sometimes the first symptoms occur suddenly because the blockage occurs suddenly—for example, when a blood clot lodges in an artery narrowed by an atheroma, causing a heart attack or stroke.

In some embodiments, an effective amount of IGF-1 reduces the area of an atherosclerotic lesion in an individual by at least about 5%, at least about 10%, at least about 15%, at least about 20%, at least about 25%, at least about 30%, at least about 35%, at least about 40%, at least about 45%, at least about 50%, or more than 50%, compared to the area of atherosclerotic lesion in an individual not treated with IGF-1.

### IGF-1

IGF-1 suitable for administration includes native IGF-1, IGF-1 variants, and analogs. Where the subject is a human subject, administration of native human IGF-1 or a variant of human IGF-1 is of particular interest.

Suitable for use in the subject methods are IGF-1 variants. IGF-1 variants can be designed that retain efficient binding to the type I IGF receptor, yet would have reduced binding to serum carrier proteins, e.g. IGFBPs. In one aspect, the design of these variants is based on the observation that insulin does not bind to serum carrier proteins. See U.S. Pat. No. 4,876,242, issued Oct. 24, 1989, herein expressly incorporated by reference in its entirety. Evidence from synthetic, insulin-like two chain analogs suggests that amino acids of IGF-1 responsible for carrier protein binding are in the B region of IGF-1. Therefore a synthetic gene for human IGF-1 can be modified to encode an IGF-1 variant in which the first 16 amino acids of hIGF-1 are replaced by the first 17 amino acids of the B chain of human insulin. The synthetic gene is then placed in a yeast recombinant DNA expression system and the peptide analog which is produced by the modified yeast cells is extracted therefrom and purified. Additional modifications of the IGF-1 molecule have been carried out leading to additional analogs, all of which have substantial IGF-1 type I receptor binding and reduced binding to serum carrier proteins.

Other IGF-1 variants and analogs well known in the art are also suitable for use in the subject methods. Such variants include, for example, the variant having resides 1-69 of authentic IGF-1, further described in WO 96/33216, and the two-chain IGF-1 superagonists which are derivatives of the naturally occurring single-chain IGF-1 having an abbreviated C domain, further described in EP 742,228. IGF-1 analogs are of the formula: BCⁿ,A wherein B is the B domain of IGF-1 or a functional analog thereof, C is the C domain of IGF-1 or a functional analog thereof, n is the number of amino acids in the C domain and is from about 6 to about 12 amino acids, including about 8 to about 10 amino acids, and A is the A domain of IGF-1 or a functional analog thereof.

Also suitable for use in the subject methods are functional mutants of IGF-1 that are well known in the art. Such functional mutants include those described in Cascieri et al. (1988, Biochemistry 27:3229-3233), which discloses four mutants of IGF-1, three of which have reduced affinity to the Type I IGF receptor. These mutants are: (Phe²³, Phe²⁴, Tyr²⁵)IGF-1 (which is equipotent to human IGF-1 in its affinity to the Types 1 and 2 IGF and insulin receptors), (Leu²⁴)IGF-1 and (Ser²⁴)IGF-1 (which have a lower affinity than IGF-1 to the human placental Type I IGF receptor, the placental insulin receptor, and the Type I IGF receptor of rat and mouse cells), and desoctapeptide (Leu²⁴)IGF-1 (in which the loss of aromaticity at position 24 is combined with the deletion of the carboxyl-terminal D region of hIGF-1, which has lower affinity than (Leu²⁴)IGF-1 for the Type I receptor and higher affinity for the insulin receptor). These four mutants have normal affinities for human serum binding proteins.

Also suitable for use with the subject methods include structural analogs of IGF-1 well known in the art. Such structural analogs include those described in Bayne et al. (1988, J Biol Chem 264:11004-11008), which discloses three structural analogs of IGF-1: (1-62)IGF-1, which lacks the carboxyl-terminal 8-amino-acid D region of IGF-1; (1-27,Gly⁴,38-70)IGF-1, in which residues 28-37 of the C region of IGF-1 are replaced by a four-residue glycine bridge; and (1-27,Gly⁴,38-62) IGF-1, with a C region glycine replacement and a D region deletion. Peterkofsky et al. (1991, Endocrinology, 128: 1769-1779) discloses data using the Gly⁴ mutant of Bayne et al., supra. U.S. Pat. No. 5,714,460 refers to using IGF-1 or a compound that increases the active concentration of IGF-1 to treat neural damage.

Other structural analogs include those described in Cascieri et al. (1989, J Biol Chem, 264: 2199-2202) discloses three IGF-1 analogs in which specific residues in the A region of IGF-1 are replaced with the corresponding residues in the A chain of insulin. The analogs are: (Ile⁴¹, Glu⁴⁵, Gln⁴⁶, Thr⁴⁹, Ser⁵⁰, Ile⁵¹, Ser⁵¹, Tyr⁵⁵, Gln⁵⁶)IGF-1, an A chain mutant in which residue 41 is changed from threonine to isoleucine and residues 42-56 of the A region are replaced; (Thr⁴⁹, Ser⁵⁰, Ile⁵¹)IGF-1; and (Tyr⁵⁵, Gln⁵⁶)IGF-1.

### IGF-1 Dosage

Selection of the therapeutically effective dose can be determined (e.g., via clinical trials) by a skilled artisan, such as a clinician or a physician, based upon the consideration of several factors which will be known to one of ordinary skill in the art. Such factors include, for example, the particular form of IGF-1, and the compound's pharmacokinetic parameters such as bioavailability, metabolism, half-life, and the like, which is established during the development procedures typically employed in obtaining regulatory approval of a pharmaceutical compound. Further factors in considering the dose include the disease or condition to be treated, the benefit to be achieved in a subject, the subject's body mass, the subject's immune status, the route of administration, whether administration of the compound or combination therapeutic agent is acute or chronic, concomitant medications, and other factors known by the skilled artisan to affect the efficacy of administered pharmaceutical agents.

In some embodiments, the total pharmaceutically effective amount of IGF-1 administered parenterally per dose will be in the range of about 10 μg/kg/day to about 400 μg/kg/day, including about 20 μg/kg/day to about 200 μg/kg/day, such as, about 40 μg/kg/day to about 100 μg/kg/day, of subject body weight, although, this will be subject to a great deal of therapeutic discretion. Exemplary doses for adults are in the range of about 10 μg/kg/day to about 160 μg/kg/day, e.g., from about 10 μg/kg/day to about 20 μg/kg/day, from about 20 μg/kg/day to about 30 μg/kg/day, from about 30 μg/kg/day to about 50 μg/kg/day, from about 50 μg/kg/day to about 60 μg/kg/day, from about 60 μg/kg/day to about 80 μg/kg/day, from about 80 μg/kg/day to about 100 μg/kg/day, from about 100 μg/kg/day to about 120 μg/kg/day, from about 120 μg/kg/day to about 140 μg/kg/day, or from about 140 μg/kg/day to about 160 μg/kg/day. Other doses of interest for adults are in the range of about 10 μg/kg/day to about 180 μg/kg/day, or about 60 μg/kg/day to about 200 μg/kg/day, e.g., from about 60 μg/kg/day to about 80 μg/kg/day, from about 80 μg/kg/day to about 100 μg/kg/day, from about 100 μg/kg/day to about 120 μg/kg/day, from about 120 μg/kg/day to about 140 μg/kg/day, from about 140 μg/kg/day to about 160 μg/kg/day, from about 160 μg/kg/day to about 180 μg/kg/day, or from about 180 μg/kg/day to about 200 μg/kg/day. In some embodiments of particular interest, 20 μg/kg/day to 240 μg/kg/day IGF-1 is administered to the subject. The IGF-1 may be administered by any means suitable, including injections (single or multiple, e.g., 1-4 per day) or infusions. In certain embodiments, the IGF-1 is administered once or twice per day by subcutaneous bolus injection. In some embodiments, about 30 μg/kg to about 50 μg/kg IGF-1 is administered to the subject by subcutaneous bolus injection once or twice per day. If a slow release formulation is used, typically the dosages used (calculated on a daily basis) will be less, up to one-half of those described above.

In some embodiments, the IGF-1 therapy of the invention may be administered to the patient in the form of a single or twice daily administration of an immediate release formulation of IGF-1.

### Routes of Administration

Administration of the pharmaceutical compositions of the invention includes, but is not limited to, oral, intravenous infusion, subcutaneous injection, intramuscular, topical, depo injection, implantation, time-release mode, intracavitary, intranasal, inhalation, intralesional, intraocular, immediate release, and controlled release. The pharmaceutical compositions of the invention also may be introduced parenterally, transmucosally (e.g., orally), nasally, rectally, intravaginally, sublingually, submucosally, or transdermally. In some embodiments, administration is parenteral, i.e., not through the alimentary canal but rather through some other route via, for example, intravenous, subcutaneous, intramuscular, intraperitoneal, intraorbital, intracapsular, intraspinal, intrasternal, intra-arterial, or intradermal administration. In some embodiments, the administering of IGF-1 is by other than direct administration to the pericardial space. In other embodiments, the administering of IGF-1 is by systemic administration. In some of these embodiments, IGF-1 is systemically administered by subcutaneous bolus injection.

The skilled artisan can appreciate the specific advantages and disadvantages to be considered in choosing a mode of administration. Multiple modes of administration are encompassed by the invention. For example, an IGF-1 protein is administered by subcutaneous injection, whereas a combination therapeutic agent is administered by intravenous infusion. Moreover, administration of one or more species of IGF-1 proteins, with or without other therapeutic agents, may occur simultaneously (i.e., co-administration) or sequentially. For example, an IGF-1 protein is first administered to increase sensitivity to subsequent administration of a second therapeutic agent or therapy. In another embodiment, the periods of administration of one or more species of IGF-1 protein, with or without other therapeutic agents may overlap. For example, an IGF-1 protein is administered for 7 days, and a second therapeutic agent is introduced beginning on the fifth day of IGF-1 protein treatment, and treatment with the second therapeutic agent continues beyond the 7-day IGF-1 protein treatment. The IGF-1 can also be administered intermittently in a cyclical manner as described in U.S. Pat. No. 5,565,428.

### IGF-1 Formulations and Dosage Forms

The present invention further provides methods for treating a subject having atherosclerosis using a pharmaceutical composition of IGF-1, and a pharmaceutically acceptable carrier. Suitable pharmaceutically acceptable carriers include essentially chemically inert and nontoxic pharmaceutical compositions that do not interfere with the effectiveness of the biological activity of the pharmaceutical composition. Examples of suitable pharmaceutical carriers include, but are not limited to, saline solutions, glycerol solutions, ethanol, N-(1(2,3-dioleyloxy)propyl)-N,N,N-trimethylammonium chloride (DOTMA), diolesylphosphotidylethanolamine (DOPE), and liposomes. Such pharmaceutical compositions should contain a therapeutically effective amount of the compound, together with a suitable amount of carrier so as to provide the form for proper administration to the subject. The formulation should suit the mode of administration. For example, oral administration requires enteric coatings to protect the compounds of the invention from degradation within the gastrointestinal tract. In another example, the compounds of the invention may be administered in a liposomal formulation, particularly for nucleic acids, to shield the compounds from degradative enzymes, facilitate transport in circulatory system, and effect delivery across cell membranes to intracellular sites.

In another embodiment, a pharmaceutical composition comprises an IGF-1 protein, and/or one or more therapeutic agents; and a pharmaceutically acceptable carrier. In one embodiment, a pharmaceutical composition, comprising a IGF-1 protein, with or without other therapeutic agents; and a pharmaceutically acceptable carrier, is at an effective dose.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In some embodiments, the composition is formulated in accordance with routine procedures as a pharmaceutical composition adapted for subcutaneous injection or intravenous administration to humans. Typically, pharmaceutical compositions for subcutaneous injection or intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the composition is to be administered by infusion, it can be dispensed with an infusion bottle, bag, or other acceptable container, containing sterile pharmaceutical grade water, saline, or other acceptable diluents. Where the composition is administered by injection, an ampule of sterile water for injection or saline can be provided so that the ingredients may be mixed prior to administration.

In certain embodiments, the formulation for IGF-1 is that described in U.S. Pat. No. 5,681,814. This formulation is as follows: about 2 to about 20 mg/ml of IGF-1, about 2 to about 50 mg/ml of an osmolyte, about 1 to about 15 mg/ml of at least one stabilizer, and a buffer (such as an acetic acid salt buffer, or sodium acetate) in an amount such that the composition has a pH of about 5 to about 5.5. Optionally, the formulation may also contain a surfactant, e.g., in an amount of from about 1 mg/ml to about 5 mg/ml, e.g., from about 1 mg/ml to about 3 mg/ml.

In some embodiments, the osmolyte is an inorganic salt at a concentration of about 2-10 mg/ml or a sugar alcohol at a concentration of about 40 to about 50 mg/ml, the stabilizer is benzyl alcohol, phenol, or both, and the buffered solution is an acetic acid salt buffered solution. In further embodiments, the osmolyte is an inorganic salt, such as sodium chloride.

In yet further embodiments, the formulation includes about 8 to about 12 mg/ml of IGF-1, about 5 to about 6 mg/ml of sodium chloride, benzyl alcohol as the stabilizer in an amount of about 8 to about 10 mg/ml and/or phenol in an amount of about 2 to about 3 mg/ml, and about 50 mM sodium acetate buffer so that the pH is about 5.4. Optionally, the formulation contains polysorbate as a surfactant in an amount of about 1 to about 3 mg/ml.

Pharmaceutical compositions adapted for oral administration may be provided, for example, as capsules or tablets; as powders or granules; as solutions, syrups or suspensions (in aqueous or non-aqueous liquids); as edible foams or whips; or as emulsions. Tablets or hard gelatine capsules may comprise, for example, lactose, starch or derivatives thereof, magnesium stearate, sodium saccharine, cellulose, magnesium carbonate, stearic acid or salts thereof. Soft gelatine capsules may comprise, for example, vegetable oils, waxes, fats, semi-solid, or liquid polyols, etc. Solutions and syrups may comprise, for example, water, polyols and sugars.

An active agent intended for oral administration may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location.

Pharmaceutical compositions adapted for parenteral administration include, but are not limited to, aqueous and non-aqueous sterile injectable solutions or suspensions, which may contain antioxidants, buffers, bacteriostats and solutes that render the pharmaceutical compositions substantially isotonic with the blood of an intended recipient. Other components that may be present in such pharmaceutical compositions include water, alcohols, polyols, glycerine and vegetable oils, for example. Compositions adapted for parenteral administration may be presented in unit-dose or multi-dose containers, for example, sealed ampules and vials, and may be stored in a freeze-dried (lyophilized) condition requiring the addition of a sterile liquid carrier, e.g., sterile saline solution for injections, immediately prior to use. Extemporaneous injection solutions and suspensions may be prepared from sterile powders, granules and tablets. Such pharmaceutical compositions should contain a therapeutically or cosmetically effective amount of a compound which increases IGF-1 blood levels, together with a suitable amount of carrier so as to provide the form for proper administration to the subject. The formulation should suit the mode of administration.

Pharmaceutical compositions adapted for transdermal administration may be provided as discrete patches intended to remain in intimate contact with the epidermis for a prolonged period of time. Pharmaceutical compositions adapted for topical administration may be provided as, for example, ointments, creams, suspensions, lotions, powders, solutions, pastes, gels, sprays, aerosols or oils. A topical ointment or cream can be used for topical administration to the skin, mouth, eye or other external tissues. When formulated in an ointment, the active ingredient may be employed with either a paraffinic or a water-miscible ointment base. Alternatively, the active ingredient may be formulated in a cream with an oil-in-water base or a water-in-oil base.

Pharmaceutical compositions adapted for topical administration to the eye include, for example, eye drops or injectable pharmaceutical compositions. In these pharmaceutical compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for nasal administration may comprise solid carriers such as powders (e.g., having a particle size in the range of 20 to 500 microns). Powders can be administered in the manner in which snuff is taken, i.e., by rapid inhalation through the nose from a container of powder held close to the nose. Alternatively, pharmaceutical compositions adopted for nasal administration may comprise liquid carriers such as, for example, nasal sprays or nasal drops. These pharmaceutical compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided, for example, as pessaries, tampons, creams, gels, pastes, foams or spray formulations.

Suppositories generally contain active ingredients in the range of 0.5% to 10% by weight. Oral formulations can contain 10% to 95% active ingredient by weight.

In yet another embodiment, IGF-1 may be administered using long-acting IGF-1 formulations that either delay the clearance of IGF-1 from the site or cause a slow release of IGF-1 from, e.g., an injection or administration site. The long-acting formulation that prolongs IGF-1 plasma clearance may be in the form of IGF-1 complexed, or covalently conjugated (by reversible or irreversible bonding) to a macromolecule such as a water-soluble polymer selected from PEG and polypropylene glycol homopolymers and polyoxyethylene polyols, i.e., those that are soluble in water at room temperature. See, e.g., U.S. Pat. No. 5,824,642, hereby expressly incorporated by reference in its entirety. Alternatively, the IGF-1 may be complexed or bound to a polymer to increase its circulatory half-life. Examples of polyethylene polyols and polyoxyethylene polyols useful for this purpose include polyoxyethylene glycerol, polyethylene glycol, polyoxyethylene sorbitol, polyoxyethylene glucose, or the like. The glycerol backbone of polyoxyethylene glycerol is the same backbone occurring in, for example, animals and humans in mono-, di-, and triglycerides. The polymer need not have any particular molecular weight. In some embodiments, the molecular weight is between about 3500 and 100,000, or between 5000 and 40,000. In some embodiments, the PEG homopolymer is unsubstituted, but it may also be substituted at one end with an alkyl group. An exemplary alkyl group is a C1-C4 alkyl group, e.g., a methyl group. In some embodiments, the polymer is an unsubstituted homopolymer of PEG, a monomethyl-substituted homopolymer of PEG (mPEG), or polyoxyethylene glycerol (POG) and has a molecular weight of about 5000 to 40,000.

For parenteral administration, in one embodiment, the IGF-1 is formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, in some embodiments, the formulation does not include oxidizing agents and other compounds that are known to be deleterious to polypeptides.

The formulation can be prepared by contacting the IGF-1 uniformly and intimately with liquid carriers or finely divided solid carriers or both. Then, if necessary, the product is shaped into the desired formulation. In some embodiments, the carrier is a parenteral carrier; and in some embodiments, the carrier is a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or non-ionic surfactants such as polysorbates, poloxamers, or PEG; and/or neutral salts, e.g., NaCl, KCl, MgCl₂, CaCl₂, etc.

The IGF-1 can be formulated in such vehicles at a concentration of from about 0.1 mg/mL to about 100 mg/mL, or from about 1 mg/ml to about 10 mg/mL, and at a pH of about 4.5 to 8. Full-length IGF-1 can be formulated at a pH about 5-6, and des(1-3)-IGF-1 can be formulated at a pH about 3.2 to 5. It will be understood that use of certain of the foregoing excipients, carriers, or stabilizers will result in the formation of IGF-1 salts.

In addition, the IGF-1, for example the full-length IGF-1, may be formulated together in an appropriate carrier vehicle to form a pharmaceutical composition. In one embodiment, the buffer used for formulation will depend on whether the composition will be employed immediately upon mixing or stored for later use. If employed immediately after mixing, a mixture of full-length IGF-1 can be formulated in mannitol, glycine, and phosphate, pH 7.4. If this mixture is to be stored, it is formulated in a buffer at a pH of about 6, such as citrate, with a surfactant that increases the solubility at this pH, such as 0.1% polysorbate 20 or poloxamer 188. The final preparation may be a stable liquid or lyophilized solid.

An “osmolyte” refers to an isotonic modifier or osmotic adjuster that lends osmolality to the buffered solution. Osmolality refers to the total osmotic activity contributed by ions and nonionized molecules to a solution. Examples include inorganic salts such as sodium chloride and potassium chloride, mannitol, polyethylene glycols (PEGs), polypropylene glycol, glycine, sucrose, glycerol, amino acids, and sugar alcohols such as mannitol known to the art that are generally regarded as safe (GRAS). An exemplary osmolyte for use herein is sodium chloride or potassium chloride.

The “stabilizer” is any compound that functions to preserve the active ingredients in the formulation, i.e., IGF-1, so that it does not degrade or otherwise become inactive over a reasonable period of time or develop pathogens or toxins that prevent their use. Examples of stabilizers include preservatives that prevent bacteria, viruses, and fungi from proliferating in the formulation, anti-oxidants, or other compounds that function in various ways to preserve the stability of the formulation.

For example, quaternary ammonium salts are useful stabilizers in which the molecular structure includes a central nitrogen atom joined to four organic (usually alkyl or aryl) groups and a negatively charged acid radical. These salts are useful as surface-active germicides for many pathogenic non-sporulating bacteria and fungi and as stabilizers. Examples include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of stabilizers include aromatic alcohols such as phenol and benzyl alcohol, alkyl parabens such as methyl or propyl paraben, and m-cresol. An exemplary stabilizer for use herein is phenol or benzyl alcohol.

The stabilizer is included in a stable liquid form of the IGF-1 formulation, but not in a lyophilized form of the formulation. In the latter case, the stabilizer is present in the bacteriostatic water for injection (BWFI) used for reconstitution. The surfactant is also optionally present in the reconstitution diluent.

The “inorganic salt” is a salt that does not have a hydrocarbon-based cation or anion. Examples include sodium chloride, ammonium chloride, potassium chloride, magnesium chloride, calcium chloride, sodium phosphate, calcium phosphate, magnesium phosphate, potassium phosphate, ammonium phosphate, sodium sulfate, ammonium sulfate, potassium sulfate, magnesium sulfate, calcium sulfate, etc. In some embodiments, the cation is sodium and the anion is chloride or sulfate. For example, in some embodiments, the inorganic salt is potassium chloride or sodium chloride.

The “surfactant” acts to increase the solubility of the IGF-1 at a pH about 4-7. In some embodiments, the surfactant is a nonionic surfactant such as a polysorbate, e.g., polysorbates 20, 60, or 80, a poloxamer, e.g., poloxamer 184 or 188, or any others known to the art that are GRAS. In some embodiments, the surfactant is a polysorbate or poloxamer, e.g., a polysorbate, e.g., polysorbate 20.

The “buffer” may be any suitable buffer that is GRAS and confers a pH of 5-6 on the IGF-1 formulation. Examples include acetic acid salt buffer, which is any salt of acetic acid, including sodium acetate and potassium acetate, succinate buffer, phosphate buffer, citrate buffer, or any others known to the art to have the desired effect. An exemplary buffer is sodium acetate, optionally in combination with sodium citrate.

The final formulation, if a liquid, can be stored at a temperature of about 2°-8° C. for up to about four weeks. Alternatively, the formulation can be lyophilized and provided as a powder for reconstitution with water for injection that is stored as described for the liquid formulation.

IGF-1 to be used for therapeutic administration can be sterile. Sterility is readily accomplished by filtration through sterile filtration membranes (e.g., 0.2 micron membranes). Therapeutic IGF-1 compositions can be placed into a container having a sterile access port, for example, an intravenous solution bag or vial having a stopper pierceable by a hypodermic injection needle.

The IGF-1 can be stored in unit or multi-dose containers, for example, sealed ampoules or vials, as an aqueous solution, or as a lyophilized formulation for reconstitution. As an example of a lyophilized formulation, 10-mL vials are filled with 5 mL of sterile-filtered it (w/v) aqueous IGF-1 solution, and the resulting mixture is lyophilised. The infusion solution is prepared by reconstituting the lyophilized IGF-1 using bacteriostatic Water-for-Injection.

The formulation containing IGF-1 can be made by many different methods. One method comprises mixing about 2-20 mg/ml of IGF-1, about 2-50 mg/ml of an osmolyte, about 1-15 mg/ml of at least one stabilizer, and a buffer (e.g., an acetic acid salt buffer, e.g., sodium acetate) in an amount such that the composition has a pH of about 5-5.5. The osmolyte, stabilizer, and buffer, and the compounds within these categories are defined above. Optionally, the formulation may also contain a surfactant selected from the types described above, e.g., in an amount of from about 1 mg/ml to about 5 mg/ml, or from about 1 mg/ml to about 3 mg/ml.

In some embodiments, the osmolyte is an inorganic salt at a concentration of about 2-10 mg/ml or a sugar alcohol at a concentration of about 40-50 mg/ml, the stabilizer is benzyl alcohol, phenol, or both, and the buffered solution is an acetic acid salt buffered solution. In some embodiments, the osmolyte is an inorganic salt, e.g., sodium chloride.

In an exemplary formulation, the amount of IGF-1 is about 8 mg/ml to about 12 mg/ml, the amount of sodium chloride is about 5 mg/ml to about 6 mg/ml, the stabilizers are benzyl alcohol in an amount of about 8 mg/ml to about 10 mg/ml and/or phenol in an amount of about 2 mg/ml to about 3 mg/ml, and the buffer is about 50 mM sodium acetate so that the pH is about 5.4. Optionally, the formulation contains polysorbate as a surfactant in an amount of about 1 mg/ml to about 3 mg/ml.

In one embodiment, a pharmaceutical composition of the invention is delivered by a controlled-release or sustained release system. For example, the pharmaceutical composition may be administered using intravenous infusion, an implantable osmotic pump, a transdermal patch, liposomes, or other modes of administration. In one embodiment, a pump may be used (See, e.g., Langer, 1990, Science 249:1527-33; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the compound can be delivered in a vesicle, in particular a liposome (See, e.g., Langer, Science 249:1527-33 (1990); Treat et al., 1989, in Liposomes in the Therapy of Infectious Disease and Cancer, Lopez-Berestein and Fidler (eds.), Liss, New York, pp. 353-65; Lopez-Berestein, ibid., pp. 317-27 International Patent Publication No. WO 91/04014; U.S. Pat. No. 4,704,355). In another embodiment, polymeric materials can be used (See, e.g., Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, 1953, J. Macromol. Sci. Rev. Macromol. Chem. 23:61; Levy et al., 1985, Science 228:190; During et al, 1989, Ann. Neurol. 25:351; Howard et al., 1989, J. Neurosurg. 71:105).

Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (see Sidman et al., 1983, Biopolymers, 22:547-556), poly(2-hydroxyethyl methacrylate) (Langer et al., 1981, J. Biomed Mater Res, 15:167-277), and Langer, 1982, Chem Tech, 12; 98-105), ethylene vinyl acetate (Langer et al., supra) or poly-D-(−)-3-hydroxybutyric acid (EP 133,988). Sustained-release IGF-1 compositions also include liposomally entrapped IGF-1. Liposomes containing IGF-1 are prepared by methods known per se: DE 3,218,121; Epstein et al., 1985, Proc Natl Acad Sci USA, 82:3688-3692; Hwang et al, 1980, Proc Natl Acad Sci USA, 77: 4030-4034; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mol percent cholesterol, the selected proportion being adjusted for the optimal IGF-1 therapy.

In yet another embodiment, a controlled release system can be placed in proximity of the target. For example, a micropump may deliver controlled doses directly into the brain, thereby requiring only a fraction of the systemic dose (See, e.g., Goodson, 1984, in Medical Applications of Controlled Release, vol. 2, pp. 115-138). In other embodiments, IGF-1 is delivered at or near the heart, e.g., into a coronary artery or other coronary blood vessel.

In some embodiments, IGF-1 is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are often used because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are often used due to their generally more consistent, controlled release over time. Osmotic pumps are in some embodiments used due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the Synchromed infusion pump (Medtronic).

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiment, it may be desirable to administer the pharmaceutical composition of the invention locally to the area in need of treatment by other than a drug eluting stent; this may be achieved, for example, and not by way of limitation, by local infusion during surgery, topical application (e.g., in conjunction with a wound dressing after surgery), injection, by means of a catheter, by means of a suppository, or by means of an implant. An implant can be of a porous, non-porous, or gelatinous material, including membranes, such as sialastic membranes, or fibers.

### Combination Therapy

In another aspect, the IGF-1 regimen of the invention can be modified to include the use of additional agents for treating atherosclerosis or an atherosclerotic disease event, e.g., stroke, heart attack, heart disease including congestive heart failure, peripheral artery occlusive disease, and the like. Additional agents suitable for use in conjunction with the IGF-1 therapy of the invention include agents that increase total IGF-1 levels in the blood or enhance the effect of the IGF-1. In one embodiment, these additional reagents generally allow an excess of blood IGF-1 over the amount of IGFBPs in blood or the IGF-1 to be released from IGFBPs, and include growth-promoting agents.

The agent can be co-administered sequentially or simultaneously with the IGF-1 administration provided in the method of the invention, and may be administered in the same, higher, or a lower dose than if used alone depending on, such factors as, for example, the type of reagent used, the purpose for which the reagent and compound are being used, and clinical considerations. In addition, other means of manipulating IGF-1 status, such as regimens of diet or exercise, are also considered to be combination treatments as part of this invention.

In another embodiment, IGF-1 is appropriately administered together with any one or more of its binding proteins, for example, IGFBP-1, IGFBP-2, IGFBP-3, IGFBP-4, IGFBP-5, or IGFBP-6. Without being bound by a mechanism, co-administration of IGF-1 and an IGFBP may provide a greater response than IGF-1 alone by increasing the half-life of IGF-1.

A binding protein suitable for use is IGFBP-3, which is described in U.S. Pat. No. 5,258,287 and by Martin and Baxter, 1986, J Biol Chem, 261: 8754-8760. This glycosylated IGFBP-3 protein is an acid-stable component of about 53 Kd on a non-reducing SDS-PAGE gel of a 125-150 Kd glycoprotein complex found in human plasma that carries most of the endogenous IGFs and is also regulated by GH.

The administration of the IGF binding protein with IGF-1 may be accomplished by the method described in U.S. Pat. No. 5,187,151. Briefly, the IGF-1 and IGFBP are administered in effective amounts by subcutaneous bolus injection in a molar ratio of from about 0.5:1 to about 3:1, including about 0.75:1 to about 2:1, such as about 1:1.

One or more additional active agents, for example but not limited to anti-atherosclerotic agents, may be used in combination with the IGF-1 therapy of this invention in a single dosage formulation, or may be administered to the patient in a separate dosage formulation, which allows for concurrent or sequential administration of the active agents. The additional active agent or agents can be lipid altering compounds such as HMG-CoA reductase inhibitors, or agents having other pharmaceutical activities, or agents that have both lipid-altering effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (see U.S. Pat. No. 4,342,767) (ALTOPREV™); simvastatin (see U.S. Pat. No. 4,444,784) (ZOCOR™); dihydroxy open-acid simvastatin, particularly the ammonium or calcium salts thereof; pravastatin (PRAVACHOL™), particularly the sodium salt thereof (see U.S. Pat. No. 4,346,227); fluvastatin (LESCOL™), particularly the sodium salt thereof (see U.S. Pat. No. 5,354,772); atorvastatin (LIPITOR™), particularly the calcium salt thereof (see U.S. Pat. No. 5,273,995); nisvastatin (PITAVA™), also referred to as NK-104 (see PCT international publication number WO 97/23200); and rosuvastatin (CRESTOR™) (also known as ZD4522, see U.S. Pat. No. 5,260,440).

Additional active agents which may be employed in combination with IGF-1 include but are not limited to 5-lipoxygenase inhibitors, HMG-CoA synthase inhibitors; cholesterol ester transfer protein (CETP) inhibitors, for example JTT-705 and CP529,414; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein fibrinogen receptor antagonists and aspirin; human peroxisome proliferator activated receptor gamma (PPARγ) agonists including the compounds commonly referred to as glitazones for example troglitazonE (REZULIN™), pioglitazone (ACTOS™) and rosiglitazone (AVANDIA™), including those compounds included within the structural class known as thiazolidinediones as well as those PPARγ agonists outside the thiazolidinedione structural class; PPARα agonists such as clofibrate (ATROMID-S™), fenofibrate including micronized fenofibrate, and gemfibrozil; PPAR dual α/γ agonists such as 5-[(2,4-dioxo-5-thiazolidinyl)methyl]-2-methoxy-N-[[4-(trifluoromethyl)ph-enyl]methyl]-benzamide, known as KRP-297; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and β-carotene; β-blockers; angiotensin II antagonists such as Losartan (COZAAR™); angiotensin converting enzyme inhibitors such as enalapril (VASOTEC™) and CAPTOPRIL™; calcium channel blockers such as nifedipine (ADALAT™, PROCARDIA™) and diltiazem (CARDIZEM™, DILACOR™, TIAZAC™); endothelin antagonists such as bosentan, tezosentan, sitaxsentan, enrasentan and ambrisentan; agents that enhance ABC1 gene expression; FXR and LXR ligands including both inhibitors and agonists; bisphosphonate compounds such as alendronate sodium; and cyclooxygenase-2 inhibitors such as rofecoxib (VIOXX™) and celecoxib (CELEBREX™).

Cholesterol absorption inhibitors can also be used in combination with IGF-1 of the present, invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240; WO 00/20623, WO 97/45406, WO 97/16424, WO 97/16455, and WO 95/08532. The most notable cholesterol absorption inhibitor is ezetimibe (ZETIA™), also known as 1-(4-fluorophenyl)-3(R)-[3(S)-(4 fl-uorophenyl)-3-hydroxypropyl)]-4(S)-(4-hydroxyphenyl)-2-azetidinone, described in U.S. Pat. Nos. 5,767,115 and 5,846,966.

IGF-1 may also be administered in conjunction with one or more additional agents such as anti-inflammatory agents (e.g., non-steroidal anti-inflammatory drugs (NSAIDs; e.g., detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate) and steroids (e.g., cortisone, dexamethasone, hydrocortisone, methylprednisolone, prednisolone, prednisone, triamcinolone)), antibacterial agents (e.g., azithromycin, clarithromycin, erythromycin, roxythromycin, gatifloxacin, levofloxacin, amoxicillin, or metronidazole), platelet aggregation inhibitors (e.g., abciximab, aspirin, cilostazol, clopidogrel, dipyridamole, eptifibatide, ticlopidine, or tirofiban), anticoagulants (e.g., dalteparin, danaparoid, enoxaparin, heparin, tinzaparin, or warfarin), or antipyretics (e.g., acetaminophen).

Surgical treatment is also envisioned as a combination therapy in conjunction with administration of IGF-1. For example, balloon angioplasty can open up narrowed vessels and promote an unproved blood supply. In addition, a metallic stenting element can be inserted and used to permanently maintain the walls of the vessel treated in its extended opened state. Vascular stents are small mesh tubes made of stainless steel or other metals and are used to prop open the weak inner walls of atherosclerotic arteries. They are often used in conjunction with balloon angioplasty to prevent restenosis after the clogged arteries are treated. The blood supply to the heart muscle can also be restored through a vein graft bypass. Large atheromatous and calcified arterial obstructions can be removed by endarterectomy, and woven plastic tube grafts can replace entire segments of diseased peripheral vessels.

These secondary therapeutic agents may be administered within 14 days, 7 days, 1 day, 12 hours, or 1 hour of administration of IGF-1, or simultaneously therewith. The additional therapeutic agents may be present in the same or different pharmaceutical compositions as IGF-1. When present in different pharmaceutical compositions, different routes of administration may be used. For example, a second agent may be administered orally, while IGF-1 may be administered by intravenous, intramuscular, or subcutaneous injection.

### Kits

Kits with unit doses of the subject compounds, usually in oral or injectable doses, are provided. In such kits, in addition to the containers containing the unit doses will be an informational package insert describing the use and attendant benefits of the IGF-1 in treating atherosclerosis.

The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

The following methods and materials were used in Examples 1-4, below.

### Animals

All mice were purchased from Jackson Laboratory (Bar. Harbor, Me.) and handled according to Tulane University animal care and use regulations. Animals were fed a high-fat diet (Harlan-Teklad, Madison, Wis.; 42% fat, 1.25% cholesterol) beginning at 8 weeks of age. At the same time, infusions were begun with recombinant human IGF-1 (rhIGF-1) (1.5 mg/kg/day, 12 weeks) or sham infusions using mini-pumps (Alzet, Cupertino, Calif.). Systolic blood pressure was measured weekly using a tail-cuff system with the mice in a conscious state. A total of 38 sham-infused and 42 IGF-1 infused mice were studied.

### Real Time PCR

To determine the expression of ICAM-1, VCAM-1, eNOS, TNF-α, and smooth muscle actin, 5 μg of total RNA from 3 aortas was converted into cDNA with the First Strand cDNA Synthesis kit (Amersham) and used for the 40 cycle 2 step PCR in the Bio-Rad iCycler apparatus. PCR conditions were 95° C. for 3 min followed by 40 cycles of 95° C. for 30 seconds and 55° C. for 10 seconds. Fluorescence changes were monitored with SYBR Green PCR Supermix (Bio-Rad) after every cycle, and melting curve analysis was performed at the end of 40 cycles to verify PCR product identity. Each PCR reaction was repeated more than three times, and the average median threshold cycle values were used for analysis. Results were evaluated with the iCycler IQ Real Time Detection System Software (Bio-Rad). The names, sequences and melting temperatures (in ° C.) of the primers are provided in Table 1.

TABLE 1

| Name | Sequence (5′ to 3′) | Tm in ° C. |
|---|---|---|
| m28s-s: | TGGAATGCGAGTGCCTAG | 54.7 |
| | (SEQ ID NO: 1) | |
| m28s-as | ACCGTCCTGCTGTCTATATC | 53.5 |
| | (SEQ ID NO: 2) | |
| mTNFa-s | CGTGGAACTGGCAGAAGAG | 55.7 |
| | (SEQ ID NO: 3) | |
| mTNFa-as | GTAGACAGAAGAGCGTGGTG | 55.1 |
| | (SEQ ID NO: 4) | |
| mICAM1-s | CTTGGTAGAGGTGACTGAG | 51.6 |
| | (SEQ ID NO: 5) | |
| mICAM1-as | AAGTTGTAGACTGTTAAGGTC | 49.5 |
| | (SEQ ID NO: 6) | |
| meNOS-s | CAGCATCACCTACGACAC | 52.9 |
| | (SEQ ID NO: 7) | |
| meNOS-as | GGCTCTGTAACTTCCTTGG | 52.8 |
| | (SEQ ID NO: 8) | |
| mVCAM-s | GCCCTTTCGGAGCTGAAG | 56.2 |
| | (SEQ ID NO: 9) | |
| mVCAM-as | TTAACTGACAGCCAGCCAAG | 55.2 |
| | (SEQ ID NO: 10) | |

### Quantification of Atherosclerotic Lesions

Atherosclerosis was measured within the aortic sinus, root and throughout the aortas. Aortas were dissected from the heart to the bifurcation. The aortas were rinsed in PBS, dissected to remove connective tissue and attached fat, opened longitudinally and fixed overnight in 3.7% formaldehyde, then rinsed in PBS and stained with oil red O. Oil red O-positive lesions were analyzed using NIH Image software (NIH, Bethesda, Md., USA). A total of 29 sham and 27 IGF-1 mice were studied.

### Fluorescence-Activated Cell Sorting

Peripheral-blood mononuclear cells were isolated by Ficoll density-gradient centrifugation using pooled blood from 5 sham and 5 IGF-1 infusion mice, and stained for 20 minutes with PE-conjugated anti-mouse VEGFR2, FITC-conjugated anti-mouse Sca-1, and APC-conjugated anti-mouse c-Kit. Labeled cells were sorted with a triple-laser fluorescence-activated cell sorter (FACS; Becton-Dickinson), data were collected using a FACSCalibur machine (Becton Dickinson) and analyzed by using CELLQUEST. 10,000 events were analyzed. The experiments were repeated 3 times.

### IGF-1 Assay

Serum levels of mIGF-1 and rhIGF-1 were determined by ELISA (Diagnostic Systems Laboratories, Inc, Webster, Tex., USA) following the manufacturer's instructions after separation of binding protein by acid-ethanol extraction step. A total of 32 sham and 34 IGF-1 mice were studied.

### Cytokine Assay

Plasma levels of TNF-α and IL-6 were determined by ELISA (R&D Systems, Minneapolis, Mass., USA) following the manufacturer's instructions. A total of 32 sham and 32 IGF-1 mice were studied.

### Lipid Assay

Total, LDL and HDL cholesterol concentrations were determined using a colorimetric kit (Trinity) with cholesterol standards (Thermo) as described in Kunjanthoor et al., J. Clin. Invest. 97:1767-73 (1996).

### Statistical Analysis

Data were presented as mean SEM. Statistical analysis was performed using ANOVA, or student's t-test when appropriate. Significance was established when p<0.05.

### Example 1

### IGF-1 Infusion Reduces Atherosclerotic Lesion Formation

Atherosclerosis is an inflammatory disease, and is the single most important cause of cardiovascular disease, the leading cause of death in developed countries. In this study, the potential effects of recombinant human IGF-1(rhIGF-1) infusion on atherosclerosis were studied by using ApoE deficient mice, a well characterized animal model of atherosclerosis, exhibiting advanced lesions when fed a western diet, with a morphology similar to that seen in humans (Nakashima et al., Arterioscler. Thromb. 14:133-40 (1994)).

With the development of atherosclerosis, the arterial wall itself produces inflammatory cytokines including TNF-α and IL-6 (Barath et al., Am. J. Cardiol. 65:297-302 (1990); Rus et al., Atherosclerosis 127:263-71 (1996)), and these cytokines have been suggested as predictors of severe atherosclerosis and its complications (Tzoulaki et al., Circulation 112:976-83 (2005)). These inflammatory cytokines increase the expression of adhesion molecules such as ICAM-1 and VCAM-1 on endothelial cells, leading to the recruitment of monocytes to lesion-prone sites of large arteries (Hwang et al., Circulation 96:4219-25 (1997)).

Atherosclerosis risk factors including smoking, diabetes, oxLDL and TNF-α have profound impacts on endothelial progenitor cells (EPC) survival, growth and differentiation, and reduced levels of circulating EPC independently predict atherosclerotic disease progression (Rauscher et al., Circulation 108:457-63 (2003); Asahara et al., Science 275:964-7 (1997); Goldschmidt-Clermont et al., Circulation 112:3348-53 (2005)). Although there is no absolute molecules marker to identify EPC, in general these are positive for stem cell markers.

c-Kit and Sca-1 were used as stem cell markers and VEGFR2, also known as F1k1 for mice and KDR for humans, was used as an endothelial cell marker. The potential effects of IGF-1 infusion on atherosclerosis development and on cytokines, adhesion molecules and circulating c-Kit/VEGFR2 or Sca-1/VEGFR2 positive EPC in ApoE−/− mice were determined.

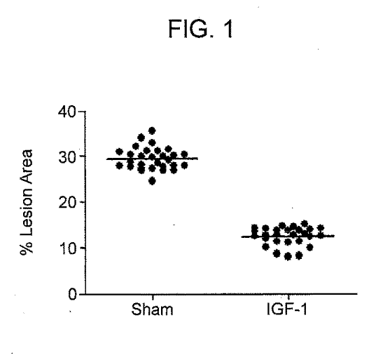

The atherosclerotic burden was determined by en face staining of the entire aorta with oil red 0 for sham infusion mice and rhIGF-1 infusion mice. It was found that rhIGF-1 infusion (1.5 mg/kg/day, 12 weeks), but not sham infusion, reduced the development of atherosclerosis (IGF-1 vs sham=12.3±2.1% vs 27.9±4.6% of total area, n=27, p<0.05) (FIG. 1)). The results show that IGF-1 infusion reduces atherosclerotic lesion formation in ApoE deficient mice. Potential mechanisms include the effects of IGF-1 to increase circulating EPC and vascular eNOS expression and to reduce circulating cytokines and vascular cytokine and adhesion molecule expression.

The development of atherosclerosis can be considered to be due to the loss of equilibrium between injury and repair. In addition, EPCs play an important role in vascular repair (Rauscher et al., Circulation 108:457-63 (2003); Asahara et al., Science 275:964-7 (1997); Xu et al., Circ. Res. 93:e76-86 (2003)). Thus, the ability of IGF-1 to increase circulating EPCs in the ApoE mouse could play an important role hi its anti-atherosclerotic effects. EPCs have been shown to incorporate into sites of neovascularization or home to sites of endothelial denudation, and differentiate into endothelial cells (Kalka et al., PNAS 97:3422-7 (2000). Moreover, it has been reported that incorporated EPC may promote vascular regeneration via a paracrine manner by releasing factors, which support local angiogenesis and mobilize tissue residing progenitor cells (Urbich et al., J. Mol. Cell. Cardiol. 39:733-42 (2005)). Thus, the IGF-1 induced increase in EPCs results in prevention of vascular lesion formation via multiple mechanisms.

### Example 2

### IGF-1 Infusion Does Not Alter Systolic Blood Pressure, Body Weight, or Endogenous IGF-1 levels

It was next determined whether administration of IGF-1 alters systolic blood pressure, body weight, or endogenous IGF-1 levels in the treated subjects. The results showed that IGF-1 did not alter systolic blood pressure, body weight, or mouse IGF-1 levels (IGF-1 vs sham=333.0±27.1 vs 334.7±23.9 ng/ml, n=38, p=NS), whereas circulating levels of rhIGF-1 were 393.2±13.1 ng/ml resulting in a 2.1 fold increase (n=38, p<0.01) in total IGF-1 in IGF-1 infused animals compared with sham (Table 2). The results also showed that elevated cholesterol levels in ApoE mice (compared to wild type mice) were not altered after IGF-1 infusion (IGF-1 vs sham=967.2±22.7 vs 965.3±22.6 mg/dl, n=32, p=NS). Levels of LDL (IGF-1 vs sham=357.4±10.3 vs 356.1±8.6 mg/dl, n=32, p=NS), and HDL (IGF-1 vs sham=67.4±4.3 vs 66.5±5.2 mg/dl, n=32, p=NS) were also not altered after IGF-1 infusion (Table 2). These data show that the atheroprotective effects of IGF-1 infusion were not due to elimination of the hypercholesterolemic source of vascular injury in these mice.

TABLE 2

| | Sham | IGF-1 |
|---|---|---|
| | Infusion | Infusion |
| mIGF-1 | 321 ± 23 | 312 ± 34 |
| | (ng/ml) | (ng/ml) |
| rhIGF-1 | — | 378 ± 31* |
| | | (ng/ml) |
| LDL | 356.1 ± 8.6 | 357.4 ± 10.3 |
| | (mg/dl) | (mg/dl) |
| HDL | 66.5 ± 5.2 | 67.4 ± 4.3 |
| | (mg/dl) | (mg/dl) |
| Cholesterol | 965.3 ± 32.6 | 967.7 ± 22.7 |
| | (mg/dl) | (mg/dl) |

### Example 3

### Effect of IGF-1 Infusion on Gene Expression

Using quantitative real-time PCR, genes that were relevant to atherosclerosis development and progression were also studied, including inflammation markers (TNF-α), endothelial dysfunction markers (ICAM-1, VCAM-1, eNOS) and smooth muscle cell markers (α-actin). The results show that IGF-1 infusion resulted in a significant increase in eNOS mRNA levels in aorta compared with controls (6.3±0.6 fold, p<0.01), but a decrease in mRNA levels of TNF-α (4.2±0.5 fold, p<0.01), ICAM-1 (3.1±0.6 fold, p<0.02), and VCAM-1 (1.8±0.2 fold, p<0.05) in aorta compared with controls. However, mRNA levels of smooth muscle cell marker (α-actin) was not significantly altered (p=NS).

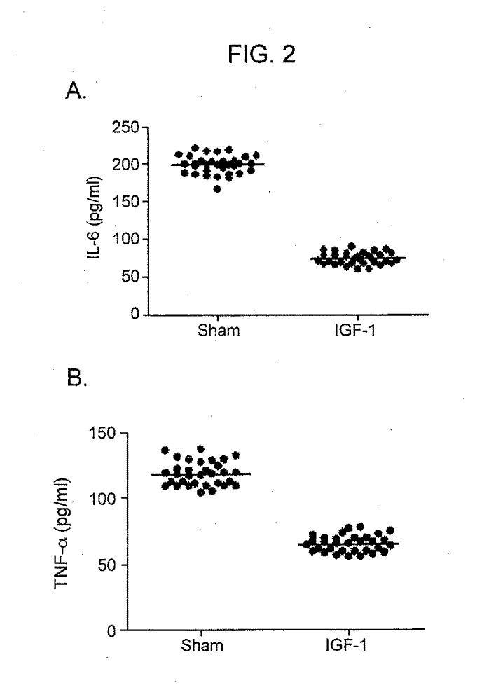

In addition, the results show that IGF-1 infusion markedly decreased circulating levels of IL-6 (IGF-1 vs sham=67.9±5.4 vs 205.4±10.4 pg/ml, p<0.001) (FIG. 2, Panel A) and TNF-α (IGF-1 vs sham=62.7±3.8 vs 120.8±11.6 pg/ml, p<0.01) (FIG. 2, Panel B). To determine whether IGF-1 infusion could alter the number of circulating EPC in ApoE^{-/-} mice, FACS analysis was performed, and found that IGF-1 increased circulating c-Kit/VEGFR2, Sca-1/VEGFR2 positive cells as assessed by flow cytometry. A summary of the data is presented in Table 3 as mean±SEM (P<0.01 compared IGF-1 and sham infusion).

TABLE 3

| | Sham | IGF-1 |
|---|---|---|
| | Infusion | Infusion |
| Sca- | 1.38 ± 0.36 | 8.47 ± 0.43 |
| 1/VEGFR2 | | |
| C- | 0.53 ± 0.21 | 3.62 ± 0.29 |
| Kit/VEGFR2 | | |

The results that IGF-1 infusion decreased inflammatory cytokines without altering lipid profiles is consistent with the previous report that injection of bone marrow cells from young ApoE^{-/-} mice to ApoE^{-/-} mice had no effect on plasma cholesterol levels but suppressed the plasma levels of IL-6 and TNF-α (Rauscher et al., Circulation 112:976-83 (2003)). It has been reported that an increase in inflammatory cytokines, a signature of advanced atherosclerosis, could itself participate in vascular injury and atherosclerosis progression (Huber et al., Arterioscler. Thromb. Vase. Biol. 19:2364-7 (2001)). It is possible that “injured” blood vessels may secrete inflammatory cytokines and that IGF-1 via its ability to induce vascular repair may reduce inflammatory cytokines release. It is of note that local IGF-1 gene transfer attenuates expression of the inflammatory cytokine TNF-α in burn wounds (Spies et al., Gene. Ther. 8:1409-15 (2001)). Moreover, the results that IGF-1 infusion decreased ICAM-1 and VCAM-1 is consistent with the previous reports that IGF-1 administration delayed the onset of autoimmune EAE by down-regulating ICAM-1 gene expression in the central nervous system (Lovett-Racke et al., J. Clin. Invest. 101:1797-804 (1998)). It is of note that during ischemic reperfusion there is marked upregulation of VCAM in eNOS knockout mice (Kaminski et al., Am. J. Pathol. 164:2241-9 (2004)) and overexpression of eNOS suppresses arterial VCAM-1 expression in hypertensive rats (Li et al., Arterioscler. Thromb. Vase. Biol. 22:249-55 (2002). Thus, the decrease in VCAM and ICAM expression in aorta in response to IGF-1 could be due to effects of increased NO resulting from upregulation of eNOS.

### Example 4

### Effect of IGF-1 on Inflammatory Response and Oxidative Stress

### Materials and Methods

Materials and methods were as described, above, except where noted below.

Materials—Recombinant human IGF-1 was obtained from Tercica Inc, (San Francisco, Calif.). Anti-Sea-1 antibody conjugated with FITC and anti-Flk-1 antibody conjugated with PE were obtained from BD Biosciences (San Jose, Calif.). Dihydroethidium (DHE) was from Invitrogen (Carlsbad, Calif.). L-NAME and D-NAME were from Sigma-Aldrich (St. Louis, Mo.).

Animals—All animal experiments were performed according to protocols approved by the Institutional Committee for Use and Care of Laboratory Animals. ApoE null mice (8 weeks) were purchased from Jackson Laboratory (Bar Harbor, Me.) infused with vehicle (saline-infusion) or with recombinant human IGF-1 (1.5 mg/kg/day) and fed a Western-type diet (42% of total cal from fat; 0.15% cholesterol) for 12 weeks. Mice were kept in accordance with standard animal care requirements, housed individually and maintained on a 12-h light-dark cycle.

Preparation of Native LDL and Oxidized LDL—Native LDL (nLDL) was Separated from human plasma of healthy donors (purchased from The Blood Center, New Orleans, La.) by sodium bromide stepwise density gradient centrifugation. Oxidized LDL (OxLDL) was prepared as previously described. Briefly, an aliquot of the nLDL fraction was passed through a 10DG desalting column (Bio-Rad) to remove EDTA, and then was incubated with 5 μmol/L CuSO₄ at 37° C. for 3 h. The value for thiobarbituric acid-reactive substances (TBARS) in OxLDL was 37.2±1.2 nmol/L malondialdehyde per mg protein. TBARS was not detectable in nLDL.

Atherosclerosis Quantification—Under anesthesia, IGF-1- or saline-infused mice were perfused through the left ventricle with PBS then 4% buffered paraformaldehyde plus 5% sucrose and the heart was dissected, placed overnight in paraformaldehyde/sucrose solution and embedded in paraffin. Serial cross sections (6 μm) were taken throughout the entire aortic valve area as per Paigen et al. Paigen et al. (1987) Atherosclerosis 68(3), 231-240. Sections were routinely stained with hematoxylin and eosin (H&E). For quantification of atherosclerotic burden, full-color images of 3 sections of each aortic valve area were acquired with a DP70 digital camera operated by DP Controller software (Olympus Optical Co, Tokyo, Japan) connected to a microscope (Olympus). Thereafter, atherosclerotic lesion areas in the aortic valve were outlined manually using the count/size feature in Image-Pro plus v6.0 software (Media Cybernetics Inc). Lesion area size in pixels² was converted to mm² using a grid attached to the slide. The mean value of plaque cross-sectional areas from 3 sections was used to estimate the extent of atherosclerosis for each animal.

Atherosclerosis was also quantified using Oil Red O-stained frozen sections of aortic valves. For this measurement the portion of the heart with the attached aorta was placed on a plastic stub using OCT compound (Cell Path, UK) and serial sections (10 μm) were cut from the site where the aorta valve cusps appear to the aorta root. Every other section was collected onto a set of microscope slides, stained with Oil Red O (5 mg/ml for 12 min) and counterstained with hematoxylin. The slides were viewed and atherosclerotic lesion areas were quantified using the procedure described above.

Immunohistochemical Analysis—Serial 6 μm paraffin-embedded cross sections were taken throughout the entire aortic valve area and three sets of serial sections obtained at 60-μm intervals were used for measurement of macrophage lesion area. Every first section in each set was stained with H&E and plaque area size was estimated as described above. The second and the third section in each set were incubated with rat anti-mouse Mac-3 monoclonal antibody (1:20, M3/84 clone, BD Pharmingen) or with isotype-matched rat non-specific IgG (Abeam, Cambridge, UK) followed by incubation with biotinylated secondary antibody and avidin-peroxidase complex (Vectastain Elite ABC kit, Vector Laboratories Inc, Burlingame, USA). Sections were developed with DAB substrate kit, counterstained with hematoxylin QS and mounted in VectaMount mounting media (Vector Laboratories). The Mac-3 positive area was quantified using Image-Pro plus software and the average mean value of Mac-3-positive area obtained from 3 sets (in mm²) was used to estimate the extent of macrophage infiltration for each animal.

Aortic Superoxide Measurement—Aortic superoxide levels were measured with DHE using serial frozen sections (10 μm) obtained from the root of aorta. DHE specificity was previously validated using smooth muscle cells exposed to OxLDL. Sukhanov et al. (2006) Circ Res 99(2), 191-200. Two serial sections each from IGF-1-infused and saline-infused mice were tested in parallel. One pair of sections from IGF-1 and control mice were pre-treated with superoxide scavenger polyethylene glycol-superoxide dismutase (PEG-SOD, 100 U, Sigma) (Faraci et al. (2004) Arterioscler Thromb Vasc Biol 24(8), 1367-1373) in buffer (50 mmol/L Tris-HCL, pH 7.4) and another pair of sections was pre-incubated with buffer only. All 4 sections were stained by DHE (2 μmol/L, 45 min, 37° C.) in the dark in a humidified chamber, briefly washed and quickly imaged with a fluorescent microscope keeping the same exposure for every section. DHE fluorescence was quantified by averaging the mean fluorescence intensity within 3 identical circles placed on a plaque-free area of aortic wall using Image-Pro plus. The superoxide-induced DHE signal was expressed as PEG-SOD-inhibitable fluorescence after subtraction of the DHE signal obtained from scavenger-pretreated section. Three to five sections from each animal (n=4 for each saline- or IGF-1-infused group) were analyzed using this procedure and the average superoxide-induced DHE fluorescence was calculated.

Urine 8-isoprostane Assay—Urine was collected for 24 h in the presence of antioxidant butylated hydroxytoluene (1 mmol/L), filtered with 0.2 μm Acrodisc syringe filter (Fisher) and frozen in aliquots at −80° C. until use. Commercially available ELISA and colorimetric assay kits (Cayman Chemical, Ann Arbor, Mich.) were used to measure urine 8-isoprostane (8-IP) and creatinine levels and the 8-IP (ng) normalized to creatinine (mg) ratios calculated. All assays were repeated 3 times.

Quantitative Real Time RT-PCR—Total RNA extraction and real-time PCR protocol was as previously described. Sukhanov et al. (2006) Circ Res 99(2), 191-200. Briefly, RNA from mice aortic homogenates was isolated using TriPure Isolation Reagent (Roche Diagnostic Corp., Indianapolis, Ind.) followed by RNA purification with RNeasy mini kit (Qiagen). Two micrograms of purified RNA was converted into cDNA with the First Strand cDNA Synthesis kit (Amersham) and used for the 40-cycle two-step PCR with sequence-specific primer pairs (Table 4) in the iCycler apparatus.

TABLE 4

| Gene | Primer Sequences (5′-3′) |
|---|---|
| β-actin | Forward primer |
| | CCGTAAAGACCTCTATGCCAAC |
| | (SEQ ID NO: 11) |
| | Reverse primer |
| | ACATCTGCTGGAAGGTGGAC |
| | (SEQ ID NO: 12) |
| Interleukin-6 | Forward primer |
| | TCTCTGGGAAATCGTGGAAA |
| | (SEQ ID NO: 13) |
| | Reverse primer |
| | CCAGGTAGCTATGGTACTCCAGA |
| | (SEQ ID NO: 14) |
| eNOS | Forward primer |
| | AGAGGGATTGTGTCACTTCGTT |
| | (SEQ ID NO: 15) |
| | Reverse primer |
| | GCATATGAAGAGGGCAGCA |
| | (SEQ ID NO: 16) |

Forward and reverse primers for TNF-α are shown in Table 1 (SEQ ID NOs:3 and 4).

PCR conditions were 95° C. for 3 min followed by 40 cycles of 95° C. for 30 s and 56° C. for 10 s. Fluorescence changes were monitored with SYBR Green PCR Supermix (Bio-Rad) after every cycle, and melting curve analysis was performed at the end of 40 cycles to verify PCR product identity (0.5° C./s increase from 55-95° C. with continuous fluorescence readings). Amplicon size and reaction specificity were confirmed by 1.5% agarose gel electrophoresis. The average median threshold cycle values were used for analysis of relative expression with iCycler IQ Real Time Detection System software (Bio-Rad).

Flow Cytometry—White blood cells in EDTA-treated whole blood were immunostained with R-phycoerythrin (PE)-conjugated anti-mouse Flk-1 monoclonal antibody and fluorescein isothiocyanate (FITC)-conjugated anti-mouse Sca-1 monoclonal antibody (all from BD Biosciences) and were analyzed by flow cytometer immediately. Sca-1+/Flk-1+ cells were considered to be endothelial progenitors, and thus were counted to evaluate circulating EPC number.

Endothelial Cell Culture—Human aortic endothelial cells were obtained from Lonza (Basel, Switzerland) and maintained in EGM-2 medium with supplements (Lonza). Cells at passages 2 to 10 were used for experiments.

Quantification of Reactive Oxygen Species (ROS) Production—Generation of intracellular superoxide was measured as described by Higashi et al. (Higashi et al. (2005) J Lipid Res 46(6), 1266-1277) using DHE fluorescence. Human aortic endothelial cells were grown to confluence on a 96-well plate (FluoroNunc™ black plate, Nalge Nunc, Rochester, N.Y.) and were washed once with phenol red-free EGM medium containing serum and supplements. The cells were preincubated in the same medium containing 100 ng/mL IGF-1 and 100 μmol D-NAME or L-NAME for 1 h, and subsequently treated with 60 μg/mL native LDL or OxLDL for 2 h. ROS formation was determined by staining cells with 5 μmol/L DHE for 30 minutes. The fluorescent intensity was read directly from the culture plate at emission wavelength of 590 nm and excitation wavelength of 485 nm with Synergy HT microplate reader (BIO-TEK instruments, Winooski, Vt.).

Statistical Analysis—All numerical data are expressed as mean±SEM. Two-tailed unpaired Student t tests were performed to determine statistical significance. Differences were considered significant at P<0.05.

### Results

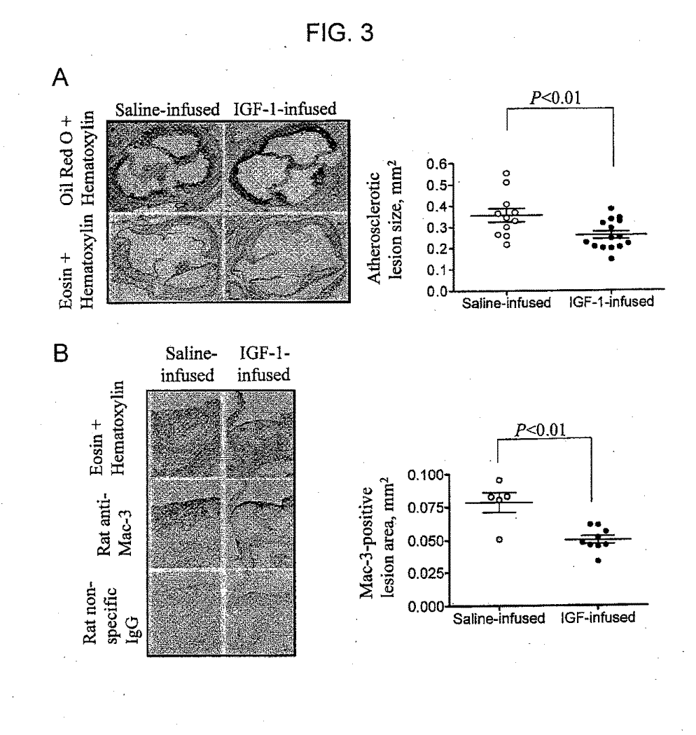

IGF-1 reduces macrophage infiltration and decreases atherosclerosis progression in ApoE^{-/-} mice—IGF-1 and saline-infused ApoE-deficient mice fed a Western diet developed early stages of atherosclerotic lesions (presence of foam cells were evident) and advanced lesions (cholesterol crystals and acellular areas were present). Morphometric analysis of atherosclerotic plaque accumulation on the intimal surface of the aortic valve revealed that IGF-1-infused mice had a 27% reduction in lesion size compared to control mice (0.259±0.018 mm² vs. 0.356±0.031 mm²) (FIG. 3, A). We estimated macrophage levels within the atherosclerotic lesions of IGF-1-infused and control mice as an index of atherosclerotic burden and also as an indicator of inflammatory responses. The plaque area staining positive for macrophages in IGF-1-infused mice was decreased by 36% compared to control (0.050±0.003 mm² vs. 0.078±0.007 mm²) (FIG. 3, B) with a slight reduction in macrophage area/lesion area ratio (20.2±1.7% vs. 25.3±1.8%, P>0.05). These data demonstrate that IGF-1 reduced macrophage infiltration and atherosclerotic plaque progression over 12 weeks, consistent with an anti-inflammatory effect.

FIGS. 3A and 3B. IGF-1 suppresses atherosclerotic plaque progression and reduces macrophage accumulation in aortic valves of ApoE-deficient mice. A-IGF-1 decreases atherosclerotic lesion size. Frozen or paraffin-embedded cross sections from saline- or IGF-infused ApoE-deficient mice were obtained throughout the aortic valve area and stained with Oil Red O and hematoxylin or H&E, respectively than imaged and quantified with Image-Pro Plus. Magnification×100. B IGF-1 reduces macrophage accumulation within atherosclerotic plaque. Three sets of paraffin-embedded serial sections were used for measurement of extent of macrophage immunopositivity per animal by staining with H&E, rat anti-mouse Mac-3 antibody and with rat non-specific IgG. Antibody-stained sections were incubated with biotinylated secondary antibody and avidin-peroxidase complex, developed with DAB substrate and counterstained with hematoxylin. Magnification×400. Aortic lesion size and Mac-3-positive lesion area are shown for each mouse from saline-infused group (empty circles) or IGF-1-infused group (solid circles). Vertical bars are mean±SEM per group.

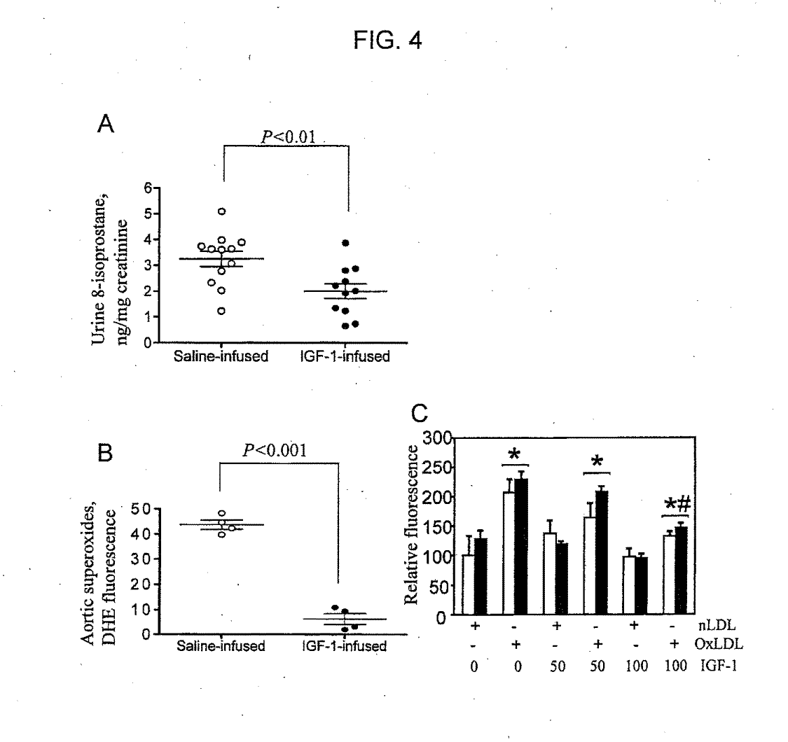

IGF-1 suppresses oxidative stress in ApoE^{-/-} mice and in cultured endothelial cells—Oxidative stress plays a critical role in atherosclerosis initiation and progression, and 8-isoprostane levels are an index of systemic oxidative stress that correlate with the severity of atherosclerosis in humans and in animal models. Patrignani et al. (2005) Biomarkers 10 Suppl 1, S24-29. It was found that IGF-1 infusion decreased urine 8-IP levels by 39% (FIG. 4A). To measure the effect of IGF-1 on aortic superoxide levels, frozen sections from the aortic roots of IGF-1-infused and control mice were stained with DUE with/without pretreatment with the superoxide-specific scavenger PEG-SOD (FIG. 4B). IGF-1 markedly suppressed superoxide levels in the aortae of ApoE-deficient mice (85% decrease compared with saline-infused mice, FIG. 4B).

It was explored whether IGF-1 could reduce oxidative stress in cultured human aortic endothelial cells. Consistent with previous reports (Rueckschloss et al. (2001) Circulation 104(15), 1767-1772, Galle et al. (2000) Nephrol Dial Transplant 15(3), 339-346) oxidized LDL induced superoxide formation in endothelial cells after 2 h of incubation. Intriguingly, co-incubation with IGF-1 suppressed OxLDL-induced superoxide generation (66% decrease with 100 ng/mL; n=4, P<0.001, FIG. 4C). L-NAME, a nitric oxide (NO) synthase inhibitor, did not significantly blunt this anti-oxidant effect of IGF-1. Superoxide levels with L-NAME tended to be higher than those with the inactive isomer D-NAME, but not significantly so.

FIGS. 4A-C. IGF-1 inhibits oxidative stress in vivo and in cultured endothelial cells. A-IGF-1 decreases urine 8-isoprostane levels, an index of systemic oxidative stress. Urine 8-isoprostane (8-IP) levels were normalized to urine creatinine levels. All assays were repeated 3 times and mean values are shown. B IGF-1 decreases aortic superoxide levels. Aortic superoxides were measured by staining aortic root sections with DHE with or without pretreatment with PEG-SOD. B-superoxide-induced DUE signal is expressed as PEG-SOD-inhibitable mean fluorescence for each mouse from saline-infused group (empty circles) or IGF-1-infused group (solid circles). Vertical bars are mean±SEM per group. C-IGF-1 reduces OxLDL-induced ROS generation in human aortic endothelial cells. Human aortic endothelial cells were incubated with indicated concentration (ng/mL) of recombinant human IGF-1 and D-NAME (empty bar; 100 μmol/L) or L-NAME (solid bar; 100 μmol/L) for 1 h and for another 2 h with nLDL or OxLDL (60 μg/mL) in addition to IGF-1 and D-/L-NAME. Superoxide generation was determined by co-incubating the cells with 5 μmol/L DHE for 30 min followed by fluorescent intensity determination. *P<0.001 vs. nLDL-treated controls (n=4). ^{#}P<0.001 vs. OxLDL and no IGF-1 treated cells (n=4).

IGF-1 downregulates vascular pro-inflammatory cytokine gene expression and increases endothelial NO synthase expression and circulating endothelial progenitor cells—IGF-1 infusion did not change body weight or total cholesterol levels (Table 5) indicating that the atheroprotective effects of IGF-1 were not due to elimination of the hypercholesterolemic source of vascular injury in ApoE-deficient mice. However, mouse (endogenous) IGF-1 levels were significantly lower in IGF-1-infused mice (P<0.001, n=7), probably due to a negative feed-back effect. The total concentration of IGF-1 (mouse IGF-1 plus human IGF-1) in IGF-1-infused mice was approximately twice that of saline-infused mice (Table 5).

TABLE 5

| | Saline infused | IGF-1 infused | P value |
|---|---|---|---|
| Body weight (g) | 25.2 ± 0.5 | 26.6 ± 0.8 | 0.15 |
| Total cholesterol (mg/dl) | 617 ± 28 | 653 ± 27 | 0.38 |
| Human IGF-1 (ng/ml) | N.D. | 487 ± 78 | |
| Murine IGF-1 (ng/ml) | 287 ± 8 | 178 ± 15 | <0.001 |
| TNF-α (pg/ml) | 23.2 ± 14.3 | 16.1 ± 5.2 | 0.65 |
| IL-6 (pg/ml) | 60.1 ± 12.7 | 69.2 ± 13.5 | 0.63 |

Table 5: for the body weight,
Saline infused: n = 13,
IGF-1 infused: n = 14;
for the other measurements,
n = 7 in each group;
N.D., not detected.

To further determine mechanisms of IGF-1's anti-atherogenic effect, tissue and circulating levels of IL-6 and TNF-α, pro-inflammatory cytokines involved in atherosclerotic lesion development, were measured. No changes were detected in serum levels of these cytokines; however aortic IL-6 and TNF-α mRNA levels were reduced 2.9-fold and 4.2-fold, respectively (Table 6), consistent with an anti-inflammatory effect of IGF-1 on the vascular wall.

TABLE 6

| mRNA expression | Saline infused | IGF-1 infused | P value |
|---|---|---|---|
| eNOS | 1.00 | 4.36 ± 0.92 | <0.05 |
| IL-6 | 1.00 | 0.34 ± 0.02 | <0.005 |
| TNF-α | 1.00 | 0.24 ± 0.15 | <0.05 |

Circulating endothelial progenitor cells (EPCs) contribute to angiogenesis in normal vascular development and are part of a vascular repair system under pathological conditions including atherosclerosis. Werner et al. (2007) Ann Med 39(2), 82-90, Xu et al. (2003) Circ Res 93(8), e76-86. Circulating EPC levels were measured in IGF-1- and saline-infused mice by assessing Sca-1+/Flk-1+ cells with FACS analysis. There was a strong trend to more circulating EPCs in IGF-1-infused mice compared to control (3.58±0.82% vs. 1.61±0.42%, P=0.06, n=8 in IGF-1-infused mice and n=7 in saline-infused mice). This result suggests that IGF-1 promotes endothelial repair by increasing circulating EPCs, potentially contributing to atheroprotection.

Endothelial nitric oxide synthase (eNOS) is the major nitric oxide-producing enzyme in the vasculature and as such can exert multiple beneficial effects including an anti-oxidant effect and atheroprotective effect. Kawashima et al. (2004) Arterioscler Thromb Vasc Biol 24(6), 998-1005. It has been reported that eNOS activity is essential for IGF-1-stimulated EPC mobilization (Thum et al. (2007) Circ Res 100(3), 434-443) and eNOS expression is inhibited in advanced atherosclerosis. Oemar et al. (1998) Circulation 97:2494. It was found that IGF-1 infusion markedly upregulated eNOS gene expression in the aorta (Table 6). This result provides initial evidence suggesting that eNOS may be involved in the anti-atherogenic effect of IGF-1.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method for treating atherosclerosis in a subject, the method comprising;
administering to a subject having atherosclerosis an effective amount of insulin like growth factor-1 (IGF-1), wherein said administering is effective to treat atherosclerosis in the subject.

2. The method of claim 1, wherein said IGF-1 is administered in a dose of about 10 μg/kg/day to 400 μg/kg/day.

3. The method of claim 1, wherein said IGF-1 is administered in a dose of about 20 μg/kg/day to 240 μg/kg/day.

4. The method of claim 1, wherein said IGF-1 is provided in a sustained release formulation.

5. The method of claim 1, wherein said IGF-1 is administered systemically to the subject.

6. The method of claim 5, wherein said IGF-1 is administered subcutaneously.

7. The method of claim 5, wherein said IGF-1 is administered to the subject by bolus injection.

8. The method of claim 1, wherein said IGF-1 is administered by a mini-pump.

9. The method of claim 8, wherein said IGF-1 is administered in a rate of about 10 μg/kg/day to 400 μg/kg/day.

10. The method of claim 1, wherein said IGF-1 is administered systemically to the subject in a sustained release formulation.
